(19) United States
(12) Patent Application Publication
(10) Pub. No.: US 2006/0058602 A1
(43) Pub. Date: Mar. 16, 2006

(54) Interstitial fluid analyzer

(76) Inventors: Krzysztof C. Kwiatkowski, College Station, TX (US); James W. Magnuson, College Station, TX (US)

Correspondence Address: STREETS & STEELE, 13831 NORTHWEST FREEWAY, SUITE 355, HOUSTON, TX, 77040, US

(21) Appl. No.: 10/920,036

(22) Filed: Aug. 17, 2004

(51) Int. Cl. A61B 5/05

(52) U.S. Cl. 600/407

(57) ABSTRACT

A device useful for measuring an analyte in the interstitial fluid of an animal comprising an array chamber having an array of one or more microprojections and a detection compartment comprising a sensor in selective fluid communication with the array chamber. Also included are two extraction electrodes for inducing electrotransport of the interstitial fluid from the animal into the array chamber. A method includes the steps of forming a plurality of microchannels through a stratum corneum layer of an epidermis of the animal, inducing electrotransport of interstitial fluid containing the analyte through the microchannels and mixing one or more materials with the interstitial fluid to form a mixture, contacting the mixture with detection electrodes and analyzing the mixture with the detection electrodes.

### BACKGROUND OF THE INVENTION

1. Field of the Invention

The present invention relates to medical devices, and more specifically, to an apparatus and method for monitoring analytes in the body.

2. Background of the Related Art

In the United States, as well as around the world, diabetes is an important health concern striking millions of diabetics. Diabetics can significantly safeguard their health and longevity by intensively monitoring their blood glucose levels in real time. Unfortunately, existing methods for monitoring blood glucose levels are limited. Therefore, there is a need for a noninvasive method to rapidly and nearly continuously measure the levels of glucose and other analytes without causing discomfort to the user or causing other harmful side effects.

Advances have been made and interest is growing in using percutaneous or transdermal sampling for such noninvasive measurements. An important goal of research in these areas is to develop methods and devices that increase the analytes' transdermal sampling rate. One method of increasing the transdermal sampling rate relies on the application of an electric current across the body surface, known as “electrotransport.” Electrotransport refers generally to the passage of an agent through a body surface, such as skin, mucous membranes, nails, and the like. The transport of the agent is induced or enhanced by applying an electrical potential, which results in the application of electric current.

The electrotransport of agents through a body surface may be attained in various manners. Well known electrotransport processes include iontophoresis, electroosmosis, and electroporation. Iontophoresis involves the electrically induced transport of charged ions. Electroosmosis involves the movement of a solvent with the agent through a membrane under the influence of an electric field. Electroporation involves the passage of an agent through pores formed by applying a high voltage electrical pulse to a membrane. In many instances, more than one of these processes may occur simultaneously to different extents.

The skin is the body's largest organ and covers the entire body. It's purpose is to serve as a protective shield against heat, light, injury and infection as well as for regulating body temperature, storing water and fat, providing sensory perception, and preventing water loss and the entry of bacteria. The skin includes three major layers: the epidermis, the dermis and the subcutaneous layer. The epidermis itself further includes three layers: the stratum corneum, the keratinocytes, and the basal layer. The epidermis is a thin layer, and contains no nerve endings, the nerve endings being found in the dermis layer.

The stratum corneum prevents the entry of most foreign substances into the body as well as the loss of fluid from the body. The stratum corneum is therefore, the major restriction to the electrotransport of agents through the skin.

Research has been conducted to improve the sampling rate obtained when using electrotransport through the skin. One method of increasing the agent transdermal sampling rate involves pre-treating the skin with a skin permeation enhancer. The term “permeation enhancer” is broadly used herein to describe a substance which, when applied to a body surface through which the agent is sampled, enhances its transdermal flux. The mechanism may involve an increase in the permeability of the body surface, or in the case of electrotransport sampling, a reduction of the electrical resistance of the body surface to the passage of the agent therethrough and/or the creation of hydrophilic pathways through the body surface during electrotransport.

The disclosure of U.S. Pat. No. 6,219,574 issued to Cormier, et al., is herein incorporated by reference in its entirety. Cormier discloses a percutaneous sampling device having a plurality of microblades that pierce the skin to increase transdermal flux of an agent and methods of its manufacture. Each of the blades typically has a width of between 25 μm and 500 μm and a length of between 20 μm and 400 μm. Cormier generally discloses that the microblades may be coupled with many different types of sampling devices, such as reverse electrotransport, passive diffusion and osmotic suction and further discloses that once the microblades pierce the skin, they are not retracted until the device is removed. Therefore, the device is typically attached to a patient and used for a 24 hour period and then removed from the patient.

In U.S. Pat. No. 5,885,211 issued to Eppstein, et. al, a method for enhancing the permeability of the skin to an analyte for diagnostic purposes or to a drug for therapeutic purposes using microporation of the stratum corneum. Eppstein discloses that the microporation may be accomplished by: ablating the stratum corneum by localized rapid heating of water that erodes the cells; puncturing the stratum corneum with a microlancet; focusing a tightly focused beam of sonic energy onto the stratum corneum; hydraulically puncturing the stratum corneum with a high pressure fluid jet; or puncturing the stratum corneum with short pulses of electricity.

U.S. Pat. No. 6,312,612, issued to Sherman, et al., discloses a microneedle array and methods of its construction from silicon and silicone dioxide compounds and is hereby incorporated by reference in its entirety. The microneedle array disclosed by Sherman may be utilized for interstitial fluid sampling/testing and for high-rate drug delivery into the body. Sherman discloses that insertion of microneedles into the stratum corneum decreases the electrical resistance of the stratum corneum by a factor of about fifty, thereby reducing the required applied voltage during iontophoresis. A blood glucose device is disclosed that extracts glucose through the skin that has been punctured by the microneedle array and subjected to iontophoresis. The glucose moves into a chamber that is filled with hydrogel and glucose oxidase which is then analyzed by a biochemical sensor.

U.S. Pat. No. 6,083,196 issued to Trautman, et al., which is hereby incorporate by reference in its entirety, discloses a device having a sheet member having a plurality of microprotrusions that penetrate the skin and a incompressible agent reservoir contacting and extending across the sheet member. The reservoir may be used to hold drugs for injections or as a reservoir for accumulating bodily fluids extracted through the skin. The Trautman device, as is generally known in the prior art, provides that the microprotrusions penetrate the skin and remains in a penetrated condition until the device is removed. In fact, the benefit offered by Trautman is that the microprotrusions are more likely to remain in the skin-piercing relationship to the skin even during and after normal patient body movement.

U.S. Pat. No. 6,050,988 issued to Zuck, discloses a device for penetrating the skin of a patient. This device has a plurality of microprotrusions, each with microprotrusions slanted in a manner as to anchor the microprotrusions in the skin after they have pierced the skin. Many of these devices of the prior art are not designed or meant to be used for long periods of time on a patient. Therefore, it is not a problem for them to leave the microprotrusions in a skin-piercing relationship to the skin.

U.S. Pat. No. 6,322,808 issued to Trautman, et al. and U.S. Pat. No. 6,091,975 issued to Daddona, et al., which are both hereby incorporated by reference in the entirety, disclose microprojections that may be used to cut microconduits through the stratum corneum.

A variety of chemicals and mechanical means have been explored to enhance transdermal flux. However, there is still a need to provide a device that is suitable for increasing transdermal flux, is low in cost, and can be manufactured in high volume production with high reproducibility, i.e., without significant variation from device to device. It would be advantageous if the device could be worn by a patient for long periods of time while monitoring an analyte of the body. Methods and devices are needed that address the problems of the existing methods for electroosmotic extraction: low volumes of extracted fluids, slow response time by the analyzers, requirements for frequent calibration, pH changes at the electrodes, and skin damage.

### SUMMARY OF THE INVENTION

The present invention provides methods and devices for measuring an analyte in the interstitial fluid of an animal. In one embodiment of an apparatus useful for measuring an analyte in the interstitial fluid of an animal, the apparatus includes an array chamber that includes an array of one or more microprojections and a detection compartment that comprises a sensor in selective fluid communication with the array chamber. The device further includes two extraction electrodes for inducing electrotransport of the interstitial fluid by providing an electrical potential across a sampling site from which the interstitial fluid is withdrawn. The electrical potential across the sampling site is induced between one of the extraction electrodes that is in electrical communication with the array chamber and the other electrode that is placed in electrical communication with the sampling site at a location adjacent to the array chamber. The device may further include an electronic control module.

The array of microprojections is adapted for transiently perforating the epidermis of the animal. The device further includes means for causing the array to transiently perforate an epidermis of the animal such as a piezoelectric stack attached to the array. Alternatively, the means may include a spring and an electromagnet attached to the array, wherein the spring pushes the array to perforate the epidermis and the electromagnet pulls the array from the epidermis or alternatively, the electromagnet pushes the array to perforate the epidermis and the spring pulls the array from the epidermis. The means for the array to transiently perforate the epidermis may be adapted to provide perforation of the epidermis to a depth of between about 50 μm and about 150 μm and preferably, the microprojections are adapted to transiently perforate the epidermis to a depth greater than the thickness of a stratum corneum layer of the epidermis but less than a total thickness of the epidermis..

The tip of the microprojections may have a diameter of between about 0.5 μm and about 5 μm, or preferably, between about 1 μm and about 2 μm and are typically made of materials selected from tungsten, platinum, silicon, gold or silver. Optionally, the microprojections are made of etched tungsten wire plated with platinum. The arrays may have a density of microprojections between about 3 microprojections per square centimeter and about 1000 microprojections per square centimeter or preferably between about 50 microprojections per square centimeter and about 500 microprojections per square centimeter.

To provide protection to the skin, the device preferably includes a salt bridge for providing electrical resistance between one of the extraction electrodes and the array chamber. Preferably, a salt bridge provides electrical resistance between both of the extraction electrodes and the sample site. Without limitation, the salt bridge typically comprises agarose gel although other suitable materials known to those having ordinary skill in the art is acceptable.

A power source provides the power for applying a potential across the extraction electrodes. Typically the power source is a battery. The device may include a switch to selectively alternate the current between the two extraction electrodes, wherein each extraction electrode selectively operates as a cathode or an anode. Preferably, the power source provides a pulsed current to the extraction electrode. The pulsed current may be in the form of a sine wave, a triangle wave, a square wave or combinations thereof. The pulsed current may be characterized as having an exponential decay. Preferably, the first of the two extraction electrodes may be made of platinum and the second electrode may typically be made of a material selected from gold, platinum, silver, palladium, graphite, or glassy carbon.

The sensor may include a working electrode, a reference electrode and a counter electrode that are each in electrical communication with the electronic control module that preferably comprises a potentiostat. Preferably, the counter electrode and the working electrode are platinum or may be selected from gold, graphite or glassy carbon. Preferably the reference electrode is an Ag/AgCl electrode.

The device may further include one or more reservoirs in selective fluid communication with the array chamber and the detection compartment and one or more micropumps for pumping the contents of the one or more reservoirs to the array chamber, the detection compartment, or combinations thereof.

The device may include additional array chambers and/or additional detection compartments. Each of the two or more array chambers comprise an array having one or more microprojections and each of the array chambers in electrical communication with either the first or the second of the two extraction electrodes. Each of the two or more detection compartments comprises a sensor in selective communication with one or more of the array chambers.

The present invention further provides methods for measuring an analyte in the interstitial fluid of an animal. In a preferred embodiment, the method includes the steps of forming a plurality of microchannels through a stratum corneum layer of an epidermis of the animal, inducing electrotransport of interstitial fluid containing the analyte through the microchannels and mixing one or more materials with the interstitial fluid to form a mixture. Additional steps may include contacting the mixture with detection electrodes and conducting amperometric analysis on the mixture with the detection electrodes.

The step of inducing electrotransport of the interstitial fluid may result in electroosmosis, reverse iontophoresis or combinations thereof. The plurality of microchannels are formed by transiently perforating the stratum corneum with microprojections. The microprojections may be arranged in one or more arrays, typically two arrays, wherein each array has one or more microprojections.

The method may furthering include the steps of separating a cathode electrode and an anode electrode from the epidermis of the animal with salt bridges, wherein the cathode electrode and the anode electrode are used in the step of electrokinetically inducing a flow of interstitial fluid and reversing polarity of the cathode electrode and the anode electrode after a performance of the step of conducting amperometric analysis. Additional steps include inducing a voltage potential across the plurality of microchannels as part of the step of inducing electrotransport of interstitial fluid.

In one preferred embodiment of a method of the present invention, the analyte is glucose and the one or more materials that are mixed with the interstitial fluid may include glucose oxidase, (dimethylaminomethyl)ferrocene and phosphate buffer. This preferred method further includes measuring the oxidation peak of (dimethylaminomethyl)ferrocene to determine glucose concentration in the interstitial fluid. Other embodiments of the present invention provide methods wherein the analyte is albumin, cholesterol, urea, a tumor metabolite or an unbound cancer drug.

The foregoing and other objects, features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIG. 1 is a schematic drawing of a microfluidic device for determining the concentration of an analyte in a patient's interstitial fluid in accordance with the present invention.

FIG. 2 is a drawing of a microscope photograph showing a 0.25 mm diameter tungsten microprojection with a tip diameter of about 1 μm.

FIG. 3 is a graph showing the signal dependence on glucose concentration in a microfluidic flow system.

FIG. 4 is a graph showing the reproducibility of the electrochemical signal upon glucose sample injections in a microfluidic flow system.

FIG. 5 is a graph showing the dependence of glucose transfer through a skin sample that has been perforated with microprojections.

### DETAILED DESCRIPTION

The present invention provides methods and devices for analyzing chemicals in the interstitial fluid of an animal. It is beneficial for measuring many different analytes of the body and is especially beneficial for use by the millions of diabetics having the need for near continuous, real time measurements of their glucose levels. The present invention provides near continuous, non-invasive and painless measurements of analytes that are contained in the interstitial fluid.

The present invention includes extracting interstitial fluid through the skin using electrotransport collection techniques enhanced by microconduits that are cut through the stratum corneum by microprojections. Cutting microconduits through the stratum corneum reduces the electrical potential that must be applied across the skin during the electrotransport extraction of the interstitial fluid. The microprojections do not penetrate the skin to the dermis layer, which is the layer of skin below the epidermis layer and contains the sensory nerve endings. Because the microprojections do not penetrate to the depth of the sensory nerve endings, the microprojections painlessly perforate the stratum corneum to provide microconduits in the skin through which the interstitial fluid may be withdrawn by using the electrotransport collection techniques.

The microprojections may be made from materials that have sufficient strength and manufacturability to produce blades, such as, glasses, ceramics, rigid polymers, metals and metal alloys. Examples of metals and metal alloys include but are not limited to stainless steel, iron, steel, tin, zinc, copper, platinum, aluminum, germanium, nickel, zirconium, titanium and titanium alloys consisting of nickel, molybdenum and chromium, metals plated with nickel, gold, rhodium, iridium, titanium, platinum, and the like. An example of glasses includes a devitrified glass such as “PHOTOCERAM” available from Corning in Corning, N.Y. Examples of polymers include but are not limited to rigid polymers such as polystyrene, polymethylmethacrylate, polypropylene, polyethylene, “BAKELITE”, cellulose acetate, ethyl cellulose, styrene/acrylonitrile copolymers, styrene/butadiene copolymers, acrylonitrile/butadiene/styrene (ABS) copolymers, polyvinyl chloride and acrylic acid polymers including polyacrylates and polymethacrylates.

While the microprojections may be made of many different types of materials, preferred materials include tungsten, platinum, silicon, gold and silver. One preferred embodiment includes microprojections made of etched tungsten wire that is plated with platinum. Another preferred embodiment includes microprojections that are made of etched silicon block plated with platinum. Methods of manufacturing arrays of microprojections are well known to those having ordinary skill in the art as may be found, for example, in an article by Jansen, et al., J. Micromech. Microeng. 5 (1995) 115-120, which is hereby fully incorporated by reference.

The microprojections typically have a diameter of between about 0.5 μm and about 5 μm at the tip. Preferably, the microprojections have a diameter of between about 1 μm and about 2 μm at the tip. Above the tip, the microprojections typically measure about 0.25 mm for microprojections made from tungsten, platinum, gold and silver or 5 μm for microprojections made from silicon but the diameter may range from between about 1 μm and about 0.5 mm.

Because the thickness of the human epidermis is between about 50 μm and about 150 μm, the length of the microprojections for human use preferably ranges between about 50 μm and about 250 μm, more preferably between about 150 μm and about 200 μm. However, since the device may be used on other animals as well, the microprojections are typically of sufficient length to penetrate the stratum corneum layer of the epidermis of a particular animal or group of animals, but preferably not so long as to penetrate into the dermis layer and make contact with the sensory nerve endings.

The present invention includes cutting a plurality of microconduits through the stratum corneum by using arrays of the microprojections. Because the microconduits that are cut through the stratum corneum significantly decrease the current and potential required by the electrokinetic transport techniques, increasing the number of microconduits that are cut through the stratum corneum decreases the current and potential requirements of the electrokinetic techniques. Preferably, the density of the microprojections in the array is between about 5 microprojections per cm² and about 1000 microprojections per cm². More preferably, the density of the microprojections is between about 50 microprojections per cm² and about 500 microprojections per cm².

The microprojections preferably do not have hooks on the tips. Hooks or barbs are often fashioned on the tips of the microprojections to anchor them to the skin and are preferred in devices that require the microprojections to remain in a skin-piercing relationship with the skin at all times. However, in a preferred embodiment of the present invention, the microprojections only transiently pierce the skin and are thereby withdrawn from the skin after each piercing. It is therefore preferred that the microprojections have smooth tips, without hooks or barbs, so that when they are withdrawn from the skin, the microprojections do not rip chunks of skin upon their withdrawal.

In a preferred embodiment, the present invention provides means for the arrays of microprojections to transiently perforate the epidermis so that the microprojections pierce the stratum corneum to form the microconduits and are then withdrawn before the electrotransport techniques are applied for extracting a sample of interstitial fluid. While not meant to be limiting to the invention, it is preferred that the time period for transiently perforating the epidermis be between about 1 second and about 10 seconds, more preferably between about 1 second and about 3 seconds.

In one preferred embodiment, the means for transiently perforating the epidermis includes a piezoelectric stack actuator attached to the array of microprojections. Piezoelectric actuators produce a small displacement when voltage is applied and are useful when very small movements are required for ultra-precise positioning. They are made by stacking piezoelectric disks or plates along an axis and they move in a linear motion along the axis of the stack when voltage is applied. Therefore, with a piezoelectric stack actuator, the voltage to be applied to the actuator may be adjusted for each individual application so that the microprojections move the exact amount required to pierce the patient's stratum corneum but not to extend into the patient's dermis layer of the skin.

Alternatively, a stored energy device, an electromagnet, manual force and combinations thereof may also be used to provide the means for transiently perforating the epidermis. For example, a spring and an electromagnet may be attached to the array so that the electromagnet pushes the array to perforate the epidermis and the spring pulls the array from the epidermis when the power to the electromagnet is removed. Likewise, the electromagnet and spring could be arranged so that the spring pushes the array to perforate the epidermis and the electromagnet pulls the array from the epidermis when the power to the electromagnet is removed. Alternatively, a user of the device could push on the array to cause microprojections to pierce the skin and a spring could pull the array from the epidermis when the pushing force is removed. Other stored energy devices, in addition to the spring, may include hydraulics, pneumatics, and other similar devices. As may be seen, there are many combinations of these forces that may be applied to the microprojections to cause them to transiently perforate the epidermis.

In one preferred embodiment, the present invention provides a device having one array on the cathode side and another array on the anode side of the extraction area. Alternatively, only one array of microprojections may be used on the side to which the interstitial fluid will flow, typically the cathode side, and the skin may remain unperforated on the opposing side, which is typically the anode side.

Optionally, multiple arrays may be used at the extraction area so that the same area of skin is not transiently perforated each time a sample of interstitial fluid is extracted. For example, four different arrays may be grouped in a block to cover the anode side and another four arrays may be grouped in a block to cover the cathode side, each of the separate arrays having its own actuator. During a particular extraction cycle, only one of the four arrays in the block is used to perforate the stratum corneum. In this manner, each of the four arrays is used only once in every four extraction cycles so that the skin in a particular extraction area is perforated at one-fourth the rate of perforation compared to an extraction area that is sampled with only one array. This will cause less irritation to the skin and allow it to recover between transient perforations.

Electrotransport techniques are used to induce greater transport of the interstitial fluid through the microconduits formed by the microprojections. Well known electrotransport processes include iontophoresis, electroosmosis, and electroporation. Iontophoresis involves the electrically induced transport of charged ions. Electroosmosis involves the movement of a solvent with the agent through a membrane under the influence of an electric field. Electroporation involves the passage of an agent through pores formed by applying a high voltage electrical pulse to a membrane. In many instances, more than one of these processes may occur simultaneously to different extents. In any case, for the present invention, each of these electrotransport techniques includes providing a potential across the sampling site that has been transiently perforated with the microprojections.

In one preferred embodiment of the present invention, two extraction electrodes, an anode electrode and a cathode electrode, provide the potential across the sampling site that is required for the electrotransport of the interstitial fluid through the microconduits that are cut through the stratum corneum. The two extraction electrodes are part of an electrotransport cell powered by a power source. Typically, the power source is a battery capable of providing between about 1 V and about 60 V. The upper voltage range is preferably between about 30 V and about 70 V and the lower voltage range is preferably between about 0.5 V and about 20 V. The power source may provide a constant current or alternatively, may provide a pulsed current. While the constant current induces the greatest flow of interstitial fluid through the microconduits, the pulsed current is better tolerated by patients and reduces the amount of skin irritation to the process. While not limiting the invention, the constant current flow rate in the electrotransport process may range between about 1.0 mA to about 4 mA, and more preferably between about 2.0 mA to about 3 mA. In a preferred embodiment of the present invention using pulsed current, the current may be oscillated, for example, at 0.5 Hz between 4.6 mA and about 0.1 mA. Alternatively, the current may be oscillated at between 0.02 Hz and 20 Hz and between about 0.1 mA and about 10 mA. When pulsed currents were used, a sine wave, a triangle wave and exponential decay wave controlled current provided better electroosmotic glucose transfer than the square wave controlled current.

The anode extraction electrode typically comprises a metal. The metal may include, without limitation, titanium, platinum, and combinations thereof. In a preferred embodiment, the anode extraction electrode is platinum. The cathode extraction electrode may typically be comprised of a metal, graphite or glassy carbon. Acceptable metals may include, without limitation, titanium, platinum, steel, gold, iron, silver, palladium, tin, nickel and combinations thereof.

While the extraction electrodes could be placed in direct contact with the skin, in a preferred embodiment the extraction electrodes are separated from contact with the skin with a salt bridge. The salt bridge typically contains, without limitation, agarose gel. The salt bridge provides electrical resistance in the circuit between the electrodes and the skin and thereby reduces the risk of burning the skin from an electrical surge.

The operation of the extraction electrodes also result in some electrolysis of water. The application of the electric field to the skin by the extraction electrodes results in the water electrolysis. The electrolysis of water results in lowering the pH at the anode and raising the pH at the cathode. The acidic pH will cause skin damage over time as the skin is exposed to the electrolysis products. By switching the polarity of the electrodes after each sampling of interstitial fluid, the deleterious effects of the electrolysis of water to the skin are neutralized. The site which is acidic after the first extraction exposed to the electrode that was the anode becomes basic after the second extraction as the electrode becomes the cathode, thereby neutralizing the pH at the skin and preventing any skin damage.

After the interstitial fluid has been extracted, the extracted sample is brought into contact with a sensor. It should be noted that a microfluidic device requires only very small quantities of extracted interstitial fluid, such as, though not limiting the invention, between about 0.5 μL and about 2 μL. Other reagents, buffers, solutions and/or chemicals may be mixed with the interstitial fluid prior to analysis by the sensor depending on the requirements of a given application. Methods that may be utilized in a sensor are well known to those having ordinary skill in the art and include, without limitation, cyclic voltammetry, AC impedance spectroscopy, AC impedance transients, and amperometry. All of these methods, as well as others known to those having ordinary skill in the are, include using an electrochemical cell and measuring the response between working, counter and/or reference electrodes. Any such method may be used in accordance with the present invention.

In one preferred embodiment of the present invention, amperometry is used as a detection method. Amperometry is an analytical method in which a constant potential is applied to an electrochemical cell, and the current response is monitored. The greater the current response, the greater the concentration of the analyte. Because the current response for a given concentration of an analyte is reproducible when using amperometry, the current response is indicative of the concentration of the analyte.

An amperometric sensor includes a reference electrode, a working electrode and a counter electrode which are all connected to a potentiostat. A constant potential is held between the reference and working electrode and the current flow is measured between the working and counter electrodes. As the concentration of the analyte changes, the measured current between the working and counter electrodes will change as the potentiostat attempts to hold the potential between the reference and working electrodes constant.

A glucose analyzer using an amperometric sensor is of particular interest. It is well known that specific glucose biosensors have been developed utilizing the enzyme glucose oxidase as a sensing biological molecule in intimate contact with a transducer. Often, the glucose oxidase is immobilized on the transducer. The transducers may work on the amperometric, potentiometric or conductometric principal.

The amperometric glucose biosensors are based on the glucose oxidase catalyzed oxidation of glucose and the production of an equivalent amount of hydrogen peroxide. Glucose concentration is then determined by the amperometric oxidation of hydrogen peroxide or oxidative peak of an electrochemical mediator, such as dimethylaminomethyl ferrocene.

A problem associated with the immobilization of the glucose oxidase on the transducer, or other enzyme or sensing molecule when testing for other analytes, is that the immobilized molecule tends to leach out from the electrode or become bound with adsorbed interference molecules, such as proteins or ions. The result is a major reduction in electrode activity that causes the signal from the electrode to decrease gradually, requiring frequent recalibration. In time, a new electrode must be installed with the sensing molecule immobilized on it to bring the electrode activity back up to an acceptable level.

To overcome these problems, a preferred embodiment of the present invention useful as a glucose monitor includes mixing a measured amount of glucose oxidase and dimethylaminomethyl ferrocene with each sample of extracted interstitial fluid. Each sample that is analyzed is then consistent with a measured amount of the reagents that were added and recalibration of the device is not required. Furthermore, since the glucose oxidase is not immobilized on the electrode, the electrode life and activity is not lessened by the leaching of the glucose oxidase from the electrode. A preferred concentration of glucose oxidase and dimethylaminomethyl ferrocene in the sample being analyzed is about 0.86 mM and about 0.80 mg/ml respectively. These concentrations of the reagents provide linear dependence of signal versus the glucose concentration over the entire range of glucose concentrations that are necessary for medical use of blood glucose levels.

The counter electrode used in the sensor may be of any suitable material known to those having ordinary skill in the art, is typically a metal, and is dependant upon the analyte. In a preferred embodiment of the present invention having a glucose sensor, the material used for the counter electrode may include, for example, gold, graphite, glassy carbon or platinum. In a preferred embodiment for a glucose sensor, platinum is used as the counter electrode.

The working electrode may also be made of many suitable materials known to those having ordinary skill in the art, is typically a metal, and is dependant upon the analyte. In a preferred embodiment of the present invention having a glucose sensor, the material may include, for example, gold, graphite, glassy carbon or platinum. In a preferred embodiment for a glucose sensor, platinum is also used as the working electrode.

The reference electrode may be made of any suitable material known to those having ordinary skill in the art, is typically a metal, and is dependant upon the analyte. In a preferred embodiment of the present invention having a glucose sensor, a Ag/AgCl electrode is used as the reference electrode.

It should be noted that a preferred electrode material is also dependant upon the analytical method used to measure the analyte. For example, the choice of electrode material may vary depending upon whether cyclic voltammetry, AC impedance spectroscopy, AC impedance transients, amperometry or other analytical method is chosen.

The present invention provides a microfluidic device for determining the concentration of an analyte in a patient's interstitial fluid. The analyte may be glucose or other analytes such as, though not limited to, albumin, cholesterol, urea, a tumor metabolite, or an unbound cancer drug. Advantageously, the device may be worn for long periods of time by the patient without damage to the patient's skin. The microfluidic device provides for non-invasive, transdermal, painless extraction of interstitial fluid through the skin followed by analysis for the analyte using, for example, amperometric or AC-current transient detection methods.

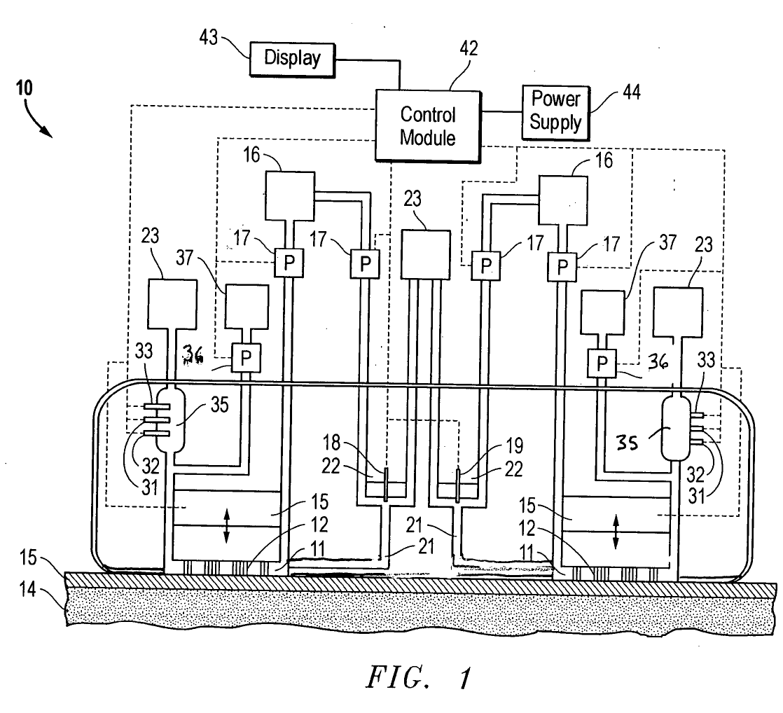

FIG. 1 is a schematic drawing of a microfluidic device for determining the concentration of an analyte in a patient's interstitial fluid in accordance with the present invention. In this preferred embodiment of the present invention, the microfluidic device 10 includes two array chambers 11 that each contains an array of microprojections 12. The microfluidic device 10 is attached to a patient's skin 14 to form a seal or floor for the array chambers 11. Attachment to the skin 14 may be accomplished by adhesives, a strap or other suitable means. Upon activation of the piezoelectric stack actuator 15, the array of microprojections 12 moves towards the patient's skin 14 to transiently pierce the epidermal layer 15 of the skin 14. The piezoelectric stack actuator 15 is calibrated so that the array of microprojections 12 preferably pierces through the stratum corneum layer of the patient's skin 14 but not into the dermis layer of the skin 14 where the sensory nerve endings are located.

A control module 42 provides computational, control and data storage capabilities for the device 10. The control module may be programmed to turn the micropumps 17, 36 on and off at the proper time, to control the piezoelectric stack actuators 15 to accurately piece the patient's skin 14, and to activate the extraction electrodes 18, 19 so that a sample of interstitial fluid may be extracted. The control module 42 further includes a potentiostat, a galvanometer or other devices that may be required for the electrochemical analysis of the analyte. Data tables are maintained -of analyte results and may be read out on a digital display 43 for viewing by the patient or a caregiver. A power supply 44, typically one or more batteries, provides the necessary power for the sensor 35, the extraction electrodes 18, 19 and other power requirements.

Liquid supply reservoirs 16 provide storage for buffer solutions, such as phosphate buffer (pH of 7), simple saline solutions, DI water or similar liquids. These liquids may be used for filling, flushing and rinsing the sensors 35, the array chambers 11, and the extraction electrode chambers 22. The liquids are pumped from the liquid supply reservoirs 16 by one or more liquid supply micropumps 17. Optionally, to reduce the number of micropumps 17 needed, microvalves (not shown) may be used to direct the flow from one micropump 17 to multiple destinations rather than using a separate micropump 17 for each destination. Micropumps are readily available from many manufacturers, such as from the Fraunhofer Institute of Munich, Germany. In some applications, a suction filter is recommended to protect the pump and to prevent the transport lines from being plugged.

To begin extraction of interstitial fluid from the patient, the extraction electrode chambers 22 are filled with a liquid from the liquid supply reservoirs 16. In a preferred embodiment, the extraction electrode chambers 22 are filled with phosphate buffer (pH=7). After the array chambers 11 have been filled with the buffer solution and the patient's skin 14 has been transiently pierced by the microprojections 12, the control module 42 directs the power supply 44 to provide a potential across the extraction electrodes 18, 19 that are immersed in the buffer solution.

The extraction electrodes 18, 19 are in electrical communication with the patient's skin 14 through the buffer solution contained in the array chambers 11 and the salt bridges 21 that provides electrical communication from the buffer solution within the array chambers 11 to the extraction electrodes 18, 19. The salt bridges 21 are preferably filled with agarose gel but may be filled with any suitable conductive material. The salt bridges 21 provide electrical resistance to in the circuit between the patient's skin 14 and the extraction electrodes 18, 19 to protect the skin 14 in the event of an electrical surge through the circuit. Furthermore, by keeping the skin 14 from direct contact with the extraction electrodes 18, 19, the skin 14 remains in a protected position from the electrodes 18, 19, allowing the patient to wear the microfluidic device 10 for long periods of time without fear of damage to the skin 14.

The extraction electrodes 18, 19 induce a current through the patient's skin 14 resulting in electroosmotic extraction of interstitial fluid from the patient into one of the array chambers 11. Because the stratum corneum has been pierced with the microprojections 12, less current is required to extract the necessary quantity of interstitial fluid by electroosmotic means. Typically, the electroosmotic force set up by the extraction electrodes 18, 19 extracts the interstitial fluid into the array chamber 11 on the cathode side. Since the extraction electrodes 18, 19 also induce some electrolysis of water, in one preferred embodiment of the present invention, the control module 42 reverses the polarity of the extraction electrodes 18, 19 after each extraction cycle so that the low pH that was generated on the anode side of the device 10 can be neutralized on the next extraction by becoming the cathode side.

After sufficient time has elapsed to collect an adequate quantity of interstitial fluid, the control module 42 turns the power off to the extraction electrodes 18, 19 to stop the electroosmotic extraction of the interstitial fluid. Typically, the extracted interstitial fluid is contained in the array chamber 11 that was on the cathode side of the extraction electrodes 18, 19.

At least one sensor 35 is provided to analyze the interstitial fluid for the analyte. In a preferred embodiment, two sensors 35 are provided that are in fluid communication with one each of the array chambers 11. Optionally, only one sensor may be used with microvalves directing the flow from the selected array chamber to the sensor. As a further option, additional sensors may be provided, with proper valving, to analyze the interstitial fluid for additional analytes. Each of the sensors 35 contains a working electrode 31, a reference electrode 32 and a counter electrode 33. The control module 42 activates the liquid supply micropump 17 to flush the interstitial fluid from the array chamber 11, thereby pushing the extracted interstitial fluid into the sensor 35. At the same time, a reagent pump 36 is activated to pump a mixture of reagents necessary for analyzing the interstitial fluid for the analyte of interest. The reagents are stored in reagent reservoirs 37 until needed. As many reagent reservoirs 37 as needed for a particular application may be provided. Optionally, the number of reagent pumps 36 may be decreased by using valving (not shown) to direct the flow to the sensors instead of using dedicated pumps for each reagent destination. The reagents mix with the interstitial fluid being flushed from the array Chamber 11 into the sensor 35.

In a preferred embodiment of the present invention that includes a sensor for determining glucose levels, the reagent pump 36 pumps a mixture of glucose oxidase and dimethylaminomethyl ferrocene to mix with the extracted interstitial fluid being flushed from the array chamber 11. In other applications involving other analytes, the reagents may be stored in one or more reagent reservoirs 37. In some applications, several reagent reservoirs may be required if the reagents cannot be mixed prior to their use in the sensor 35.

As the reagents and the extracted interstitial fluid sample flow through the sensor 35, the mixture contacts the three electrodes 31, 32, 33. As discussed above, there are several different electrochemical analytical methods that may be used to determine the analyte concentration in the interstitial fluid. In a preferred embodiment of the present invention that includes a sensor for determining glucose levels, the amperometric analytical method is used. The control module 42 uses the potentiostat, which is included in the control module 42, to measure the increase in current flowing between the working 31 and counter 33 electrodes required to maintain a constant voltage between the reference 32 and working 31 electrodes as the interstitial fluid and reagents flow past them. The control module 42 then compares the maximum measured current flow with the current flows of known concentrations of glucose, thereby determining the glucose level in the extracted interstitial fluid.

The measured concentration of the analyte in the interstitial fluid may be stored in a memory of the control module 42 and displayed on a digital display 43.

To prepare for the next cycle of extraction and measurement, the array chambers 11, the extraction electrode chambers 22 and the sensors 35 may be flushed out with liquid from the liquid storage reservoirs 16. All the liquids may be flushed to one or more waste reservoirs 23 for disposal.

### EXAMPLE 1

Microprojections were prepared using the following method. A small glass vial, having a diameter of about 2.5 cm, was filled with a solution used for electroetching wire into the microprojections. The vial was then closed with a plastic cap containing a septum. The solution used for electroetching depends upon the type of wire used for making the microprojections. For tungsten microprojections, the solution used was 0.1 M NaOH. For platinum microprojections, the solution used was saturated NaNO2 solution. For gold microprojections, the solution used contained 10 g KCN and 5 g KOH per 40 mL of water.

Three stainless steel needles were inserted through the septum so that the ends were about 5 mm above the solution level in the vial. A length of wire about 5 cm in length and having a thickness of about 0.25 mm, was inserted into the solution through the first needle to a depth of about 2 mm. A second wire was inserted through the second needle and immersed in the solution shaped in a manner to form a spiral around the wire to be electroetched. When electroetching wire made of platinum or tungsten, the wire that was used to form a spiral around the wire to be electroetched was platinum, having a thickness of about 0.5 mm. When electroetching wire made of gold, the wire that was used to form a spiral around the wire to be electroetched was also gold, having a thickness of about 0.5 mm. The third stainless steel needle was used to vent the gas produced during the electroetching.

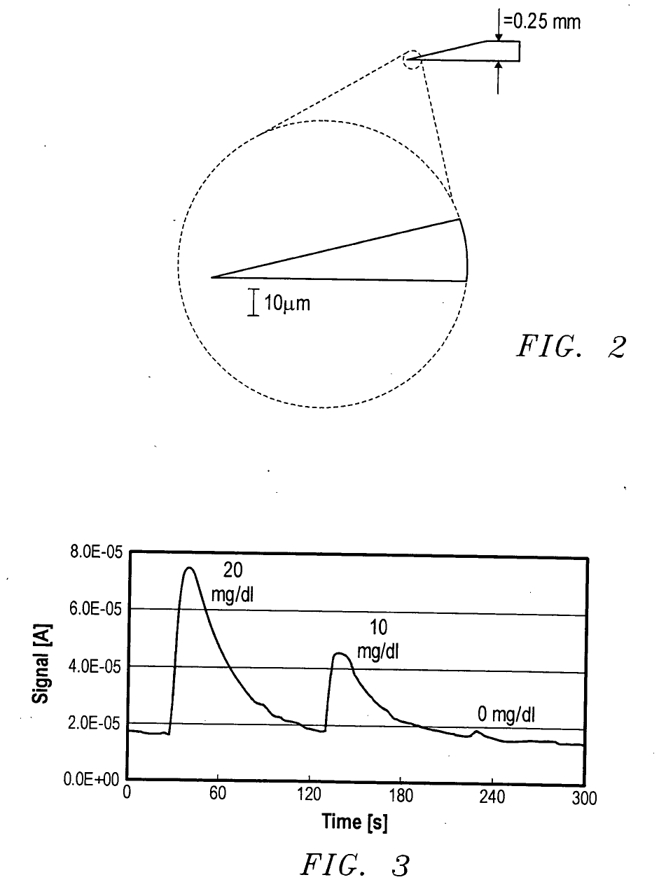

AC potentials of 10 V, 17 V, and 20 V were applied for the W, Pt and Au microprojections respectively. AC current was monitored during the electroetching. The microprojections were ready when the AC current dropped to zero. The microprojections that were formed had very sharp tips having diameters of between about 1 and about 2 micrometers. FIG. 2 is a drawing of a microscope photograph of a 0.25 mm diameter tungsten microprojection made as described above.

### EXAMPLE 2

A microfluidic flow cell was assembled. The assembly consisted of a dual glassy carbon electrode with a thin Teflon sheet, which provided the microfluidic channel structure. The channels were cut into the Teflon sheet and provided the flow from the inlet over the electrodes to the outlet. The Teflon sheet was sandwiched between two polycarbonate plates. A Ag/AgCl reference electrode was placed in the channel inlet and positioned near the working electrode.

A solution containing 0.86 mM ferrocene and 0.8 mg/mL glucose oxidase in 0.1 M phosphate buffer, pH=7, was pumped through the microfluidic flow cell using a syringe pump. Glucose solutions were prepared in 0.1 M phosphate buffer. He flow cell was tested for glucose detection using the following solutions: 0.05 mL of 20 mg/mL glucose, 0.05 mL of 10 mg/mL glucose, and 0.05 mL of phosphate buffer. The amperometric method was used for glucose detection.

The results are shown in FIG. 3. As may be seen from the graph shown in FIG. 3, the ferrocene electrochemical signal was immediate and depended upon the concentration of the injected glucose samples.

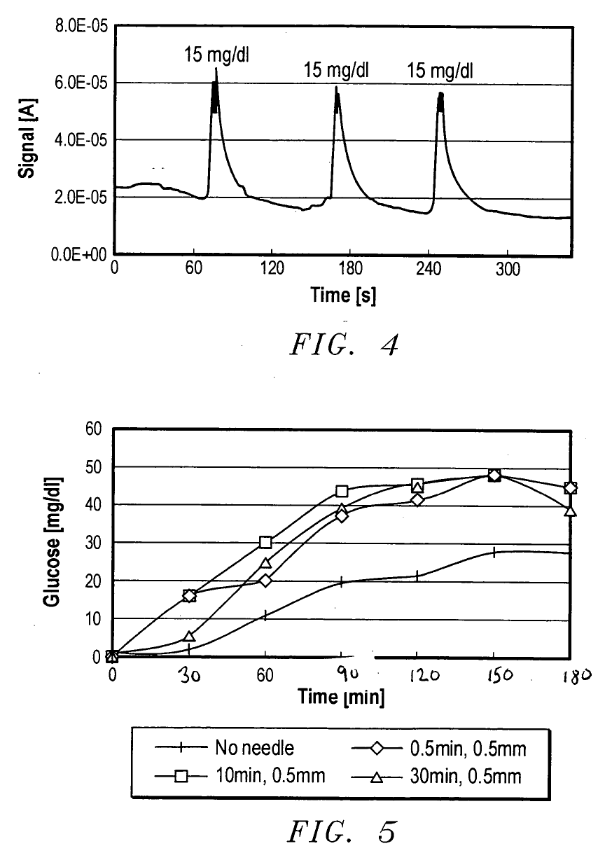

The flow cell was also tested for reproducibility using three consecutive 0.05 mL injections of 15 mg/mL glucose solution in 0.1 M phosphate buffer. The results are shown in FIG. 4. As may be seen from the graph shown in FIG. 4, the reproducibility of the glucose signal in the microfluidic flow regime was excellent. Integration of the peak areas yielded a standard error of about 0.6%.

### EXAMPLE 3

Four samples of pigskin were cut from one piece to minimize sample error. Each sample of pigskin was placed in the testing device. For each experiment, the lower compartment of the testing device was filled with 0.15 M glucose solution prepared in 0.05 M phosphate buffer, pH=7. For each data point, the cathode and the anode compartments were washed with DI water, wiped with a Kimwipe, the anode compartment was filled up with 0.1 mL of 0.8 M phosphate buffer and the cathode compartment was filled with 0.1 mL of 40 mg/dL glucose in 0.8 M phosphate buffer and charged with 0.02 mL of glacial ascetic acid. The pH in both compartments was confirmed to be neutral.

The three samples were contacted with 0.5 mm tungsten microneedle arrays for 0.5, 10, and 30 minutes, respectively during 30 minutes of electroosmosis at a current of 2 mA controlled by a Solartron Potentiostat/Galvanostat, Model 173. The fourth sample of pigskin was subjected to identical experimental conditions but was not contacted with the microprojections. The results are shown in FIG. 5.

The results shown in FIG. 5 show that electroosmotic transfer is enhanced by piercing the skin with the microprojections before starting the electroosmosis. However, as further shown by FIG. 5, the contact time of the microprojections with the skin is not significant to the glucose transfer rate. Therefore, transiently perforating the skin enhances the extraction of the glucose through the skin just as much as leaving the microprojections embedded in the skin at all times. However, by only transiently perforating the skin just prior to each extraction, potential problems involving sterility and skin irritation that are common when the microprojections remain embedded in the skin are dispensed with.

It will be understood from the foregoing description that various modifications and changes may be made in the preferred embodiment of the present invention without departing from its true spirit. It is intended that this description is for purposes of illustration only and should not be construed in a limiting sense. The scope of this invention should be limited only by the language of the following claims.

What is claimed is:

1. An apparatus for measuring an analyte in interstitial fluid of an animal, comprising:
an array chamber comprising an array of one or more microprojections;
a detection compartment comprising a sensor in selective fluid communication with the array chamber;
two extraction electrodes for inducing electrotransport of the interstitial fluid from the animal to the array chamber; and
an electronic control module.

2. The apparatus of claim 1, further comprising:
means for the array to transiently perforate an epidermis of the animal.

3. The apparatus of claim 2, wherein the means for the array to transiently perforate the epidermis comprises a piezoelectric stack attached to the array.

4. The apparatus of claim 2, wherein the means for the array to transiently perforate the epidermis comprises a spring and an electromagnet attached to the array, wherein the spring pushes the array to perforate the epidermis and the electromagnet pulls the array from the epidermis.

5. The apparatus of claim 2, wherein the means for the array to transiently perforate the epidermis comprises a spring and an electromagnet attached to the array, wherein the electromagnet pushes the array to perforate the epidermis and the spring pulls the array from the epidermis.

6. The apparatus of claim 2, wherein the means for the array to transiently perforate the epidermis are adapted to provide perforation of the epidermis to a depth of between about 50 μm and about 150 μm.

7. The apparatus of claim 1, wherein the tip of the microprojections have a diameter of between about 0.5 μm and about 5 μm.

8. The apparatus of claim 1, wherein the tip of the microprojections have a diameter of between about 1 μm and about 2 μm.

9. The apparatus of claim 1, wherein the microprojections are adapted to transiently perforate the epidermis to a depth greater than the thickness of a stratum corneum layer of the epidermis but less than a total thickness of the epidermis.

10. The apparatus of claim 1, wherein the microprojections are made of materials selected from tungsten, platinum, silicon, gold or silver.

11. The apparatus of claim 1, wherein the microprojections are made of etched tungsten wire plated with platinum.

12. The apparatus of claim 1, wherein the microprojections are made of etched silicon block plated with platinum.

13. The apparatus of claim 1, wherein each of the arrays have a density of microprojections between about 3 microprojections per square centimeter and about 1000 microprojections per square centimeter.

14. The apparatus of claim 1, wherein each of the arrays have a density of microprojections between about 50 microprojections per square centimeter and about 500 microprojections per square centimeter.

15. The apparatus of claim 1, further comprising:
a salt bridge for providing electrical resistance between one of the extraction electrodes and the array chamber.

16. The apparatus of claim 15, wherein the salt bridge comprises agarose gel.

17. The apparatus of claim 1, further comprising:
a power source for applying a potential across the extraction electrodes.

18. The apparatus of claim 17, wherein the power source is a battery.

19. The apparatus of claim 17, wherein the power source provides a pulsed current to the extraction electrodes.

20. The apparatus of claim 19, wherein the pulsed current is selected from a sine wave, a triangle wave or combinations thereof.

21. The apparatus of claim 19, wherein the pulsed current is an exponential decay.

22. The apparatus of claim 1, wherein a first of the two extraction electrodes is made of platinum.

23. The apparatus of claim 1, wherein a second of the two extraction electrodes is made of a material selected from gold, platinum, silver, palladium, graphite, or glassy carbon.

24. The apparatus of claim 1, wherein the first, extraction electrode is in electrical communication with the array chamber.

25. The apparatus of claim 24, wherein the two extraction electrodes provide an electric potential across a sampling site of the animal.

26. The apparatus of claim 1, wherein the sensor comprises a working electrode, a reference electrode and a counter electrode that are each in electrical communication with the electronic control module.

27. The apparatus of claim 26, wherein the counter electrode is platinum.

28. The apparatus of claim 26, wherein the counter electrode is selected from gold, graphite or glassy carbon.

29. The apparatus of claim 26, wherein the working electrode is platinum.

30. The apparatus of claim 26, wherein the working electrode is selected from gold, graphite or glassy carbon.

31. The apparatus of claim 26, wherein the reference electrode is an Ag/AgCl electrode.

32. The apparatus of claim 1, wherein the electronic control module comprises a potentiostat.

33. The apparatus of claim 1, further comprising:
one or more reservoirs in selective fluid communication with the array chamber and the detection compartment.

34. The apparatus of claim 1, further comprising:
one or more micropumps for pumping a contents of the one or more reservoirs to the array chamber, the detection compartment, or combinations thereof.

35. The apparatus of claim 34, wherein the one or more micropumps are started and stopped by control signals generated by the electronic control module.

36. The apparatus of claim 1, further comprising one or more waste reservoirs in selective fluid communication with the array chamber, the detection compartment, or combinations thereof.

37. The apparatus of claim 1, further comprising a switch to selectively alternate the current between the two extraction electrodes, wherein each extraction electrode selectively operates as a cathode or an anode.

38. The apparatus of claim 1, further comprising:
two or more array chambers, each array chamber comprising an array having one or more microprojections and each of the array chambers in electrical communication with either the first or the second of the two extraction electrodes.

39. The apparatus of claim 38, further comprising:
two or more detection compartments, each comprising a sensor in selective communication with one or more of the array chambers.

40. A method for measuring an analyte in interstitial fluid of an animal, comprising forming a plurality of microchannels through a stratum corneum layer of an epidermis of the animal;
inducing electrotransport of interstitial fluid containing the analyte through the microchannels;
mixing one or more materials with the interstitial fluid to form a mixture;
contacting the mixture with detection electrodes; and
conducting amperometric analysis on the mixture with the detection electrodes.

41. The method of claim 40, wherein the step of inducing electrotransport of interstitial fluid causes electroosmosis.

42. The method of claim 40, wherein the step of inducing electrotransport of interstitial fluid causes reverse iontophoresis.

43. The method of claim 40, wherein the step of forming a plurality of microchannels comprises:
transiently perforating the stratum corneum with microprojections.

44. The method of claim 43, wherein the microprojections are arranged in two arrays, wherein each array has one or more microprojections.

45. The method of claim 40, wherein the microchannels are formed to a depth less than a thickness of the epidermis.

46. The method of claim 40, wherein the microchannels are formed with a diameter less than about 5 μm.

47. The method of claim 40, wherein the microchannels are formed with a diameter less than about 1 μm.

48. The method of claim 40, further comprising:
separating a cathode electrode and an anode electrode from the epidermis of the animal with salt bridges, wherein the cathode electrode and the anode electrode are used in the step of electrokinetically inducing a flow of interstitial fluid; and
reversing polarity of the cathode electrode and the anode electrode after a performance of the step of conducting amperometric analysis.

49. The method of claim 40, wherein the step of inducing electrotransport of interstitial fluid comprises:
inducing a voltage potential across the plurality of microchannels.

50. The method of claim 40, wherein the analyte is glucose, the one or more materials comprises glucose oxidase and (dimethylaminomethyl)ferrocene.

51. The method of claim 50, wherein the one or more materials further comprises phosphate buffer.

52. The method of claim 50, wherein the step of conducting amperometric analysis comprises:
measuring the oxidation peak of (dimethylaminomethyl)ferrocene to determine glucose concentration in the interstitial fluid.

53. The method of claim 40, wherein the analyte is albumin.

54. The method of claim 40, wherein the analyte is cholesterol.

55. The method of claim 40, wherein the analyte is urea.

56. The method of claim 40, wherein the analyte is tumor metabolite.

57. The method of claim 40, wherein the analyte is an unbound cancer drug.
